(19) United States
(12) Plant Patent Application Publication
(10) Pub. No.: US 2009/0183289 P1
(43) Pub. Date: Jul. 16, 2009

(54) Hibiscus plant named Alma's Star

(50) ;

(76) Inventor: Alma N. Bodiford, Luverne, AL (US)

Correspondence Address: WINSTEAD PC, P.O. BOX 50784, DALLAS, TX, 75201, US

(21) Appl. No.: 11/974,300

(22) Filed: Oct. 12, 2007

(51) Int. Cl. A01H 5/00

(52) U.S. Cl. PLT/257

(57) ABSTRACT

A new and distinct cultivar of Hibiscus named ‘Alma's Star’ particularly characterized by its unique and distinct bloom.

### BACKGROUND OF THE INVENTION

1. Field of the Invention

The present invention relates to a new and distinct cultivar of Hibiscus plant, botanically known as Hibiscus mutabilis, and hereinafter referred to by the cultivar name Alma's Star.

2. Background

The genus Hibiscus is a wide one, comprising some 150 to 160 species, which occur mainly in tropical and sub-tropical regions. Hibiscus mutabilis, commonly known as Confederate rose, cotton rose, Dixie rose mallow and Tree lotus, is a native of China, introduced to North America in the 18^{th} century, and has been thriving in the southern landscapes for many generations.

Hibiscus mutabilis is a perennial or shrub hibiscus better known as the Confederate rose. It tends to be shrubby or treelike, though it behaves more like a perennial in northern zones. Flowers are double and are 4 to 6 inches in diameter; they open white or pink, and change to deep red. Propagation by cuttings root easiest in early spring, but cuttings can be taken at almost any time. When it does not freeze, the Confederate rose can reach heights of 12 to 15 feet with a woody trunk; however, a multi-trunk bush 6 to 8 feet tall is more typical. Once a very common plant throughout the South, Confederate rose is an interesting and attractive plant that grows in full sun or partial shade, and prefers rich, well-drained soil.

### SUMMARY OF THE INVENTION

The following represent the distinguishing characteristics of the new Hibiscus cultivar Alma's Star. The distinguishing feature of Alma's Star is its unusual bloom. Alma's Star exhibits buds of unusual size at the tips of its flowering stalks. These buds open up to present a bundle of five distinct flowers, four outer and a central cluster that produce a bundle similar to a quartered rose.

### BRIEF DESCRIPTION OF THE DRAWING

The accompanying colored photographs illustrate the overall appearance of the new cultivar, showing the colors as true as it is reasonably possible to obtain in colored reproductions of this type.

FIG. 1 depicts the distinctive blooms and buds of the Alma's Star. In the foreground is a close-up view of the upper surface of a fully expanded bloom (light pink in color) and a side perspective view of a second bloom (darker pink in color) of Alma's Star. The close-up view shows the centers (yellow coloration) of two of the five individual blooms that make up each bloom of Alma's Star. Visible in the background are two buds of unusual size . Bloom colors in the photographs may appear different from the actual colors due to light reflectance.

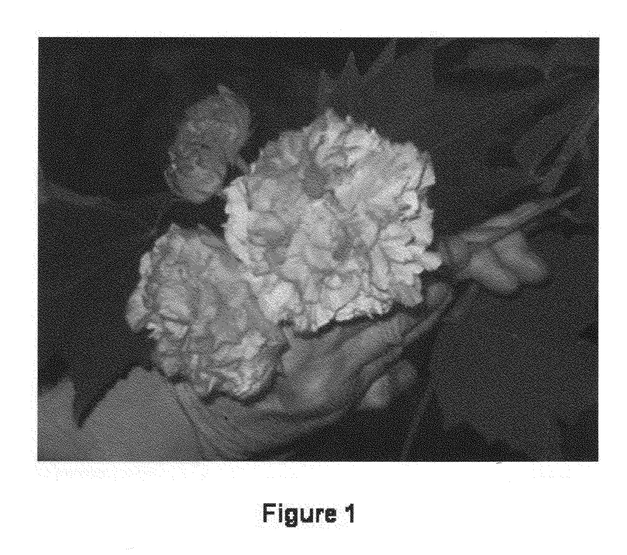

FIG. 2 shows another view of blooms of Alma's Star on a single flowering stalk. The centers (yellow coloration) of three of the five individual blooms that make up each bloom of Alma's Star are distinctly visible. Bloom colors in the photographs may appear different from the actual colors due to light reflectance.

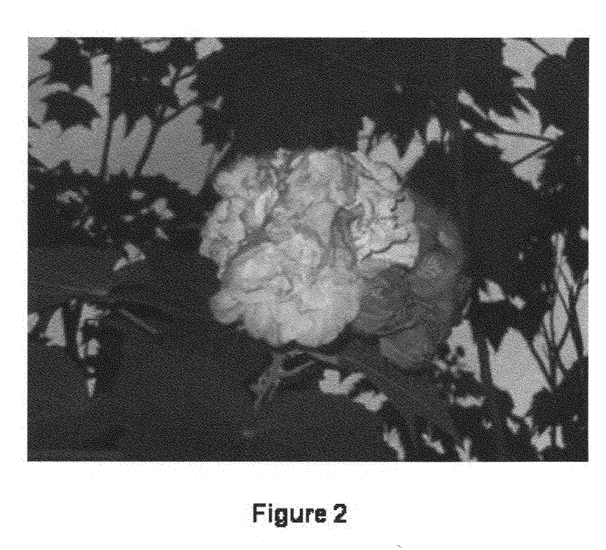

FIG. 3 shows full grown plants of Alma's Star.

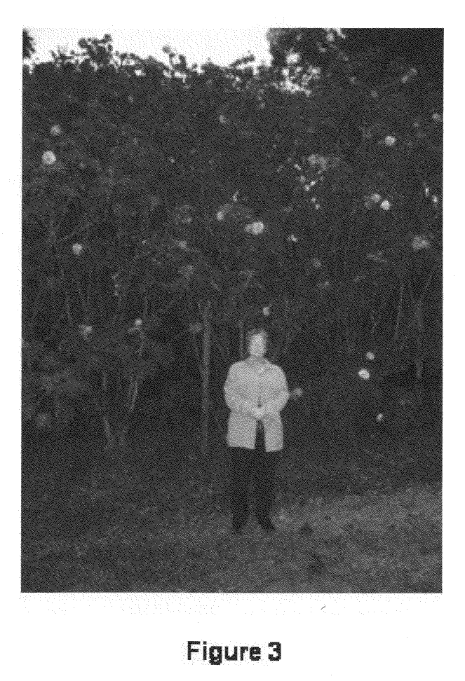

### BOTANICAL DESCRIPTION OF THE PLANT

The new cultivar is a product of an effort by the inventor to propagate garden variety Hibiscus mutabilis, in Alabama, United States of America. The objective was to restock the garden with new plants without going through seed harvesting. The new cultivar developed when natural root graftings, of rooted stalk cuttings from a Rose mallow and a Confederate rose, were planted together. Two mutated “sports” (mutated sprouts) appeared and produced unique pink flowers, each containing four or five small and separate flowers, which were sterile and produced no viable seed. The new cultivar, Alma's Star, was discovered and selected by the inventor in 1997 as a plant within the progeny of the stated cross in a cultivated area in Ala., United States of America. Asexual reproduction of the new cultivar, by top cuttings, stem cuttings and divisions taken in Alabama, United States of America, has shown that the unique features of this new Hibiscus cultivar are stable and reproduced true to type in successive generations.

The new cultivar has not been observed under all possible environmental conditions. The phenotype may vary somewhat with variations in environment such as temperature, light intensity and fertilizer rate, without, however, any variance in genotype.

The following traits have been repeatedly observed and are determined to be the unique characteristics of Alma's Star. These characteristics in combination distinguish Alma's Star as a new and distinct cultivar: Plants of the new Hibiscus have a unique and distinct bloom. The new cultivar produces buds of unusual size at the tips of its flowering stalks. These buds open up to present a bundle of five distinct flowers, four outer and a central cluster that produced a bundle similar to a quartered rose. Plants of the new Hibiscus grow at a faster rate than the confederate rose and are compact in growth habit. The new cultivar produces divisions readily giving plants a full and dense appearance. Root development on the cuttings is rapid. Typically roots are observed at 4-5 weeks in water with no additives. Roots are initiated in plain water in a well-lit area in a clear vessel. Roots on cuttings can be developed year round. Plants of the new Hibiscus have good postproduction longevity and are tolerant of low light conditions. However, best growth is in full sun. Plants of the new Hibiscus are tolerant of low winter temperatures, with the stalks surviving temperatures as low as 22 degrees Fahrenheit. Typically, the only cold injury observed is loss of leaves at hard frost. Additionally, the new cultivar is exceptionally resistant to diseases common to Hibiscus.

The following observations, measurements and comparisons describe plants grown in Luveme, Ala., Unites States of America, and conditions which closely approximate those used in horticultural practice. Plants were grown under day temperatures ranging from 60 degree to 105 degree Fahrenheit and night temperatures ranging from 22 degree to 75 degree Fahrenheit.

In the following description, color references are made to The American Hibiscus Society Color Chart except where general terms of ordinary dictionary significance are used. Botanically, the new cultivar is classified as Hibiscus mutabilis almanii Alma's Star.

Alma's Star is a naturally occurring whole plant mutation of Hibiscus mutabilis. The new cultivar is propagated by stalk or root cutting. Roots can be initiated year round. Typically time to initiate roots is 4-5 weeks. Roots are initiated in a well-lit area, in water with no additives and a clear vessel. The new cultivar tends to have deep roots with the roots being similar in appearance to those of Confederate rose.

Alma's Star grows in a day temperature range of 60 degrees to 105 degrees Fahrenheit. The new plant is tolerant of low temperatures. Apart from loss of leaves, which occurs on exposure to hard frost, the stalks survive in temperatures as low as 22 degrees Fahrenheit.

The new cultivar grows upright, multi-stemmed, outward spreading with an inverted triangle, symmetrical appearance. Numerous divisions give plants a very full, dense appearance. The new cultivar appears tall to medium, bushy, well branched and woody. It has the ability to reach a height of 20 feet from soil surface to top of leaf canopy and forms a 12 feet spread, with a rounding habit. It is tolerant of low-light but grows best in full sun. The new cultivar has a vigorous, rapid growth rate. Plants will form within 30-45 days of planting a rooted top cutting.

The new cultivar has a dense to medium foliage with leaves that are medium to large in size. The leaf color varies between light and dark green in color. The leaves are deeply lobed, undulating with serrated margins and hairy on the undersides. Leaf length, fully expanded, is about 24.13 cm and width is about 25.4 cm.

Blooms of the new cultivar are of a medium texture with partial overlapping petals. The new cultivar exhibits buds of unusual size at the tips of its flowering stalks. These buds open up to present a bundle of five distinct flowers, four outer and a central cluster. The bundle is similar to a quartered rose. The petal form is ruffled, satiny in texture and reflexed or overlapped. The bloom holds color and is sterile. The new cultivar blooms best from summer to autumn. The base petals of the bloom are pink to rosy in color. The edges of the petals are pink tinged with white. The halo is bright pink to rosy. The center is compact and hidden. The petals have no discernable veins, spots or splotches. There is no style or stigmas evident. The blooms present tinge of white streaks. The blooms bear no fragrance. Plants of the new cultivar are exceptionally resistant to diseases common to Hibiscus.

1. A new and distinct variety of Hibisus mutabilis plant named Alma's Star as described and illustrated.
